(19) United States
(12) Patent Application Publication
(10) Pub. No.: US 2018/0256385 A1
(43) Pub. Date: Sep. 13, 2018

(54) ILEOSTOMY FLOW BAND

(71) Applicant: Christoper A. McConnell, Appleton, WI (US)

(72) Inventor: Christoper A. McConnell, Appleton, WI (US)

(21) Appl. No.: 15/901,986

(22) Filed: Feb. 22, 2018

Related U.S. Application Data
(60) Provisional application No. 62/470,285, filed on Mar. 12, 2017.

(51) Int. Cl. A61F 5/449

(52) U.S. Cl. CPC A61F 5/449; A61F 2005/4495

(57) ABSTRACT

An ileostomy flow band is disclosed. The invention is comprised of a rigid, light-weight band that conforms to a user's waist and has clips allowing it to be selectively-affixed to pants waistbands. The band having a concave side facing the user cradling the pouch and protecting it from impacts, compression and deformation thereby preserving proper flow and safe urine storage inside the pouch. Other objectives of the invention include allowing an individual with an ileostomy pouch to wear a conventional belt and prevent leaks by eliminating unwanted pressure and binding on the pouch connection to the body.

### CROSS-REFERENCE TO RELATED APPLICATION

The present application is related to provisional patent application Ser. No. 62/470,285, filed on Mar. 12, 2017, and titled “ILEOSTOMY FLOW BAND”. The entire disclosure of the above patent application is hereby incorporated by reference.

### FIELD OF THE INVENTION

This invention relates in general to ileostomy bags, and more particularly, to protective supports for ileostomy bags.

### BACKGROUND OF THE INVENTION

An ileostomy is a surgical opening on the abdomen (also called a stoma) and is created by bringing a portion of the small intestine outside of the skin. Urine passes out of the ileostomy and is collected in an external, flexible plastic pouch adhered to the skin. Ileostomies are usually positioned above the groin on the right hand side of the abdomen and are fastened to the stoma by means of an adhesive wafer disc. Ileostomy pouches have been in existence since the late 1960s and doctors recommend that those who have an ileostomy not wear pant belts to limit pressure on the pouch. Many inventors have developed ways to protect these pouches and keep them properly in place during normal everyday activities.

U.S. Pat. No. 5,026,362A granted to Willett disclosed a support pocket for an ileostomy pouch that is disposed on a belt worn around a user's waist to minimize tension on the stoma connection and preventing leaks. International Patent No. WO 2004062537B1 granted to May et al. disclosed a rigid flange fitted around a stoma to improve support and minimize leaking. U.S. Pat. No. D3,696,625 granted to Kuentz disclosed a pocket for an ileostomy pouch having tabs on the top to encase the pouch. U.S. Patent No. 20100249734A1 granted to Melnick disclosed a fabric pocket with shoulder straps to support an ileostomy pouch. U.S. Pat. No. 8,226,621B2 granted to Timmons disclosed a rear belt system to hold external urine pouches outside the body for users who have undergone a nephrostomy. U.S. Pat. No. 5,643,236 granted to Hadley disclosed an under-pant fluid collection system that clips to a waistband. Several, commercial ileostomy pouch support products are currently on the market and include the Stomashield (a pouch support having an elastic waist belt), Ostomy Armor (a protective shield with nylon waist strap) and the Stealth Belt (a horizontally-orientated pouch bag within a fabric waist wrap). While many of these inventions enhance protection and support ileostomy pouches, they tend to rely on bulky, waist and shoulder straps that may leave a pouch vulnerable when clothing bunches and shifts during unusual activity such as carrying heavy loads or excessive twisting. In addition, none of the aforementioned apparatus take advantage of the support that existing articles of clothing can provide—such as from pant waist bands.

### SUMMARY OF THE INVENTION

The invention disclosed in this specification is a protective support member for an ileostomy pouch that allows stoma fluids (such as urine) to flow unimpeded into the pouch at all times. Rather than depending upon bulky, straps worn around the shoulders and waist, the invention takes advantage of trouser waistbands to keep the apparatus in position. The band is comprised of a rigid, yet slightly flexible plastic, curved to conform to a user's torso and having two clips on each end of the member that grip the pant waist firmly. Once in position, a concave shape on the inner facing creates a cradle and allows a pouch to remain undisturbed during rigorous activities and fill to its intended volume. The invention also allows articles of clothing to move laterally across the torso while keeping the pouch in tact. An objective of this invention is to protect the ileostomy pouch from excessive movement and exterior loads and minimize stoma wafer disruption in an effort to minimize leakage. Another objective is to allow users the option to wear a normal belt. Users can adjust a belt as much as needed (or tighter) to keep pants up without applying any pressure to the pouch. Another objective of this disclosure is to allow users greater freedom when using the toilet during a bowel movement. When undressing, the front clip slips off one side of the pants while the aft clip anchors the band to the other half of the pants with grips. This setup allows the band to be swung easily out of the way and then quickly re-installed.

### BRIEF DESCRIPTION OF THE DRAWINGS

For the purpose of illustrating the invention, the drawings show a form of the invention which is presently preferred. However, it should be understood that this invention is not limited to the precise arrangements and instrumentalities shown in the drawings.

FIG. 1 is a perspective view of the present invention worn by a user.

FIG. 2 is a top view of the present invention.

FIG. 3 is a top view of the present invention clipped to a pair of pants having a belt.

FIG. 4 is a front view of the present invention.

FIG. 5 is a section view of the anchor clip of the invention.

### DETAILED DESCRIPTION OF THE DRAWINGS

Embodiments and examples are described below with reference to the drawings, wherein like reference numerals are used to refer to like elements throughout. In the following description, for purposes of explanation, numerous specific details in the form of examples are set forth in order to provide a thorough understanding of the various embodiments. It will be evident, however, that these specific details are not necessary to the practice of such embodiments. In other instances, well-known structures and devices are shown in block diagram form in order to facilitate description of the various embodiments. Reference throughout this specification to “one embodiment,” or “an embodiment,” means that a particular feature, structure, or characteristic described in connection with the embodiment is included in at least one embodiment. Thus, the appearances of the phrase “in one embodiment,” or “in an embodiment,” in various places throughout this specification are not necessarily all referring to the same embodiment. Furthermore, the particular features, structures, or characteristics may be combined in any suitable manner in one or more embodiments.

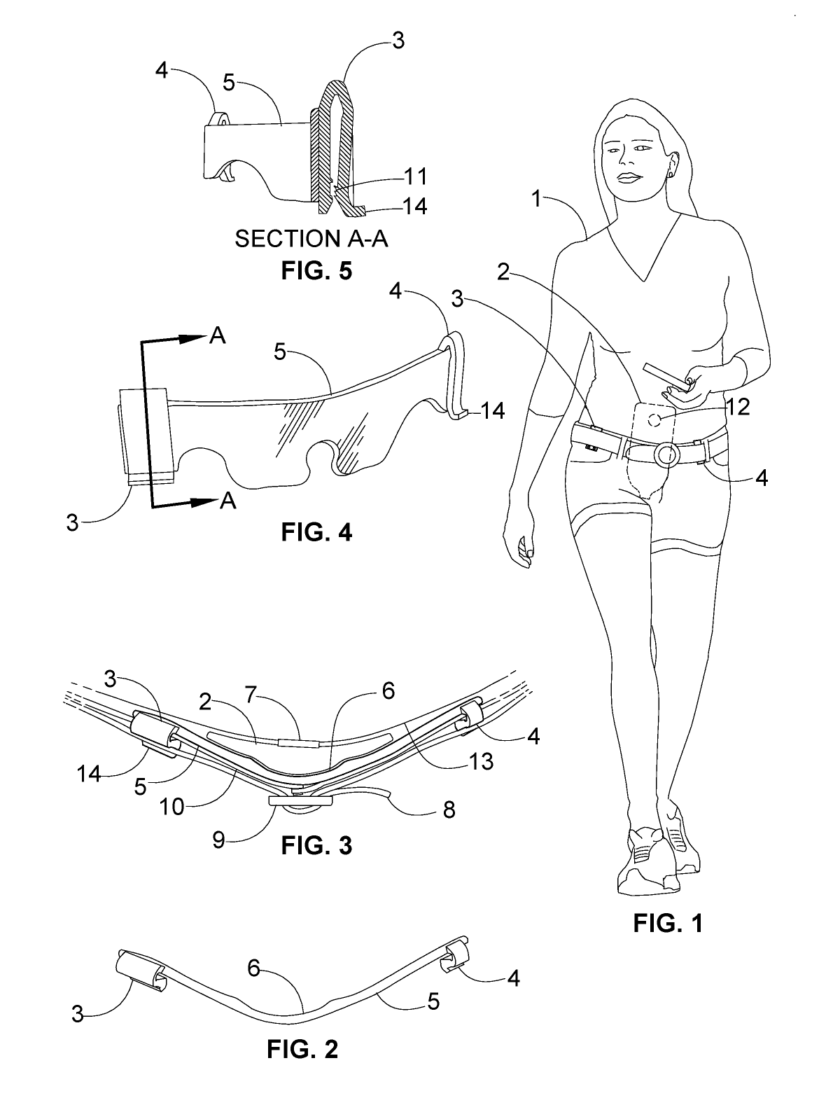

Referring now to the drawings wherein like reference characters identify corresponding or similar elements throughout several views, FIG. 1 illustrates a perspective view of one embodiment according to the present invention and shows forward slip clip 4 and aft grip clip 3 holding band 5 in place on a user. Said clips 3 and 4 being comprised of a rigid, yet bendable material such as, but not limited to plastic metal etc and being affixed to said band 5 by means of heat treatments, fasteners or adhesives etc. FIG. 1 also illustrates the invention being worn discretely behind the pants and allows for the user to wear a typical pant belt.

FIG. 2 illustrates a top view of the present invention showing said band 5 being curved in the center and swept back on each end to conform to a user's torso. Aft grip clip 3 being configured to slidably receive a typical waist band. Said clip 3 facing a direction opposite of concave 6 on another side and being disposed perpendicular to the longitudinal axis of band 5. Said clip 3 also having a teeth portion on one internal end facing band 5 to releasably grip pants. Said slip clip 4 being half the size of aft grip clip 3, being positioned on a distal end from said grip clip 3 and configured to slidably receive a pants waist band 10 (but not grip said band 10).

FIG. 3 illustrates another top view of the present invention showing band 5 surrounding a typical ileostomy pouch 2 with wafer connection 7. Clips 3 and 4 selectively affixed to pant waist 10 between a belt 8 and buckle 9 and a user's torso 13. Said clips 3 and 4 also being positioned discretely out of view of others behind belt 8. The aforementioned concave portion 6 of band 5 providing a slight gap between a user's abdomen 13 and pant waist band 10 allowing said pouch 2 to fill with fluid.

FIG. 4 illustrates a front perspective view of the present invention showing the curve in band 5 sweeping back.

FIG. 5 is a section view of the anchor clip of the invention showing grip clip 3 and the terminal end nib 14 configured to receive the bottom of a belt and preventing the device from sliding in an upward direction off of pants 10.

FIG. 6 illustrates a section view of the invention that bisects aft grip clip 3 and shows grip teeth 11 that selectively affix the invention to pants 10.

Although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes”, “having”, “has”, “with”, or variants thereof are used in either the detailed description or the claims, such terms are intended to be inclusive in a manner similar to the term “comprising.”

What is claimed is:

1. An ileostomy pouch flow band comprised of two parts: a rectangular-shaped curved plate and two clips on distal ends, said clips being affixed to a user's pant waistband allowing for proper pouch flow.

2. The ileostomy flow band of claim 1 providing protection for an ileostomy pouch—the concave shape on the inner facing creates a cradle and allows a pouch to remain undisturbed during rigorous activities and fill to its intended volume.

3. The ileostomy flow band of claim 1 providing a means to allow articles of clothing to move laterally across the torso while keeping the pouch in tact.

4. The ileostomy flow band of claim 1 providing a means to maintain ileostomy pouch connection with the body by limiting excessive movement of an ileostomy pouch while being worn by a user.

5. The ileostomy flow band of claim 1 providing a means to allow users the option to wear a normal belt, users can adjust a belt as much as needed to keep pants up without applying any pressure to the pouch.

6. The ileostomy flow band of claim 1 providing greater freedom when using the toilet during a bowel movement—during undressing, the front clip slips off one side of the pants while the aft clip anchors the band to the other half of the pants with grips.

7. The ileostomy flow band of claim 1 providing a means to prevent the invention from becoming dislodged from a belt through terminal nibs on grips.
